(19) United States
(12) Patent Application Publication
(10) Pub. No.: US 2002/0058054 A1
(43) Pub. Date: May 16, 2002

(54) Transfer-resistant cosmetic compositions comprising a non-volatile silicone compound, a non-volatile hydrocarbon-based oil, and an inert particular phase

(75) Inventor: Pascal Arnaud, L'hay Les Roses (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/941,869

(22) Filed: Aug. 30, 2001

(30) Foreign Application Priority Data
Aug. 30, 2000 (FR) 0011081

(51) Int. Cl. A61K 6/00; A61K 7/00; A61K 9/14

(52) U.S. Cl. 424/401; 424/489

(57) ABSTRACT

The present invention relates to a composition, in particular a cosmetic composition, to care for and/or make up the skin and/or integuments and/or the lips, which can be in the form of a cast product or a gel, comprising at least one non-volatile hydrocarbon-based oil with a molecular mass ranging from 230 to 420 g/Mol, a non-volatile silicone compound which is soluble or dispersible in the non-volatile hydrocarbon-based oil, and an inert particulate phase containing at least one chemically inert filler. The invention also relates to a care or make-up process for the skin, integuments and the lips using such a composition. More especially, the invention is a solid make-up product for the lips, having transfer-resistance and migration-resistance properties while at the same time being glossy and comfortable to wear over time.

### CROSS REFERENCE TO RELATED APPLICATIONS

This application claims priority to French Patent Application No. 0011081, filed on Aug. 30, 2000, and which is incorporated herein by reference in its

### BACKGROUND OF THE INVENTION

1. Field of the Invention

The present invention relates to compositions which comprise a non-volatile silicone compound and a non-volatile hydrocarbon-based oil that are mutually compatible, which is intended in particular for the cosmetics field. More especially, the invention relates to glossy transfer-resistant compositions to care for and/or make up the skin, including both human facial and body skin, the lips, the upper and lower eyelids, or integuments such as the eyelashes, the eyebrows, the nails and the hair.

The present compositions can especially be in the form of a product which is cast as a stick or as a dish, for instance lipsticks or lip balms, cast foundations, concealer products, eyeshadows or blushers; in the form of a more or less fluid paste or cream, for instance fluid foundations or lipsticks, eyeliners, mascaras, antisun compositions, colouring compositions, or artificial tanning compositions for the skin; or alternatively make-up compositions for the body or the hair.

2. DISCUSSION OF THE BACKGROUND

Make-up or care products for human skin or lips, for instance foundations or lipsticks, generally contain fatty phases such as waxes and oils, pigments and/or fillers and optionally additives, for example cosmetic or dermatological active agents. They can also contain so-called “pasty” products of soft consistency, which make it possible to obtain coloured or non-coloured pastes to be applied with a brush.

When these compositions are applied to the skin or the lips, they have the drawback of transferring, i.e., of becoming at least partly deposited, leaving traces on certain supports or surfaces with which they may come into contact, in particular such as a glass, a cup, a cigarette, an item of clothing or the skin. This results in mediocre persistence of the film applied, making it necessary to reapply the foundation or lipstick composition regularly. Moreover, the appearance of these unacceptable traces, especially on shirt collars, can put certain women off using this type of make-up.

Furthermore, these compositions have a tendency to migrate, i.e., to travel in the wrinkles and fine lines of the skin around the lips and the eyes, resulting in an unattractive effect.

In its patent application WO-A-96/40044, the company Procter & Gamble discloses lipstick compositions with transfer-resistance properties, containing a volatile oil and a non-volatile oil of the perfluoropolyether type, which are incompatible. The said patent application also discloses the enhancement of the gloss by means of the prior dispersion of an oily phase in a matrix, and the ability of this oily phase to segregate during the application of the product to the support and to migrate to the surface of the film thus deposited.

However, this system requires good dispersion of the oily phase in the matrix and can give rise to problems of stability of the product that are associated with the inevitable poor compatibility of the oily phase with the matrix.

It is moreover known that the enhancement of the gloss properties requires good dispersion of the solid particles, in particular of the pigments, in the composition. U.S. Pat. No. 5,945,092 from Revlon thus discloses the use of silicone surfactants combined with volatile oils.

Despite their efficacy, these surfactants have the drawback of being potentially irritating in particular to labial mucous membranes when their percentage in the composition is large (typically greater than 3%), and are all the more irritating when the volatile oil content is high (typically greater than 30%).

It is thus particularly advantageous to find another means for improving the gloss of transfer-resistant compositions without incurring the drawbacks mentioned above.

Furthermore, although these compositions containing volatile oils have improved “transfer-resistance” properties, they have the drawback of leaving on the lips, after these volatile oils have evaporated off, a film which very quickly becomes uncomfortable over time (sensation of dryness, tautness and discomfort), which puts a certain number of women off this type of lipstick. Furthermore, the deposit obtained is matt. Now, consumers are always on the lookout for a glossy product which is comfortable to wear throughout the day, which undergoes little or no migration in the folds of skin around the lips or the eyes, and which undergoes no or virtually no transfer.

There is thus still a need for a composition which does not suffer from the above drawbacks and which especially has good “transfer-resistance” and migration-resistance properties, even under a pronounced pressure or rubbing, while at the same time giving the deposit a more or less glossy appearance, which meets the consumer's desires, does not dry out and does not pull the skin or the lips on which it is applied, either during the application or over time.

### SUMMARY OF THE INVENTION

Accordingly, it is one object of the present invention to provide novel compositions which are useful as care or make-up compositions for keratin materials.

It is another object of the present invention to provide novel compositions which are useful as care or make-up compositions for the skin, lips, and nails.

It is another object of the present invention to provide novel compositions which are useful as care or make-up compositions for keratin materials and which are transfer resistant.

It is another object of the present invention to provide novel compositions which are useful as care or make-up compositions for keratin materials and which are migration-resistant, even under a pronounced pressure or rubbing.

It is another object of the present invention to provide novel compositions which are useful as care or make-up compositions for keratin materials and which give the deposit a more or less glossy appearance, which meets the consumer's desires.

It is another object of the present invention to provide novel compositions which are useful as care or make-up compositions for keratin materials and which do not dry out and do not pull the skin or the lips on which it is applied, either during the application or over time.

It is another object of the present invention to provide novel methods for caring for or making-up keratin materials by applying such a composition to keratin materials.

These and other objects, which will become apparent during the following detailed description have been achieved by the inventor's entirely surprising discovery that the use of a non-volatile silicone compound, an inert particulate phase and a non-volatile hydrocarbon-based oil of low molecular mass, which is compatible with the non-volatile silicone compound, in a physiologically acceptable composition and more especially a cosmetic composition, makes it possible to obtain a glossy deposit of very good staying power, which undergoes little or no transfer, does not migrate and is water-resistant, while at the same time being very pleasant to apply and to wear throughout the day. The deposit is soft and creamy.

In the context of the present invention, the term “compatible” means that the silicone compound is soluble or dispersible in the non-volatile hydrocarbon-based oil, at elevated temperature and at room temperature, and forms a phase which is homogeneous to the naked eye. Preferably, the silicone compound is soluble in the hydrocarbon-based oil.

### DETAILED DESCRIPTION OF THE PREFERRED EMBODIMENTS

Thus, in one embodiment, the present invention provides care or make-up compositions for keratin materials, comprising:

(a) at least one non-volatile hydrocarbon-based oil with a molecular mass ranging from 230 to 420 g/Mol;

(b) at least one non-volatile silicone compound which is compatible with the non-volatile hydrocarbon-based oil; and

(c) an inert particulate phase.

Advantageously, the non-volatile hydrocarbon-based oil, also referred to as a non-volatile solvent, of low molecular mass, has a molecular mass ranging from 240 to 350 g/Mol, preferably from 240 to 300 g/Mol and better still from 240 to 280 g/Mol. Also advantageously, the composition is free of volatile silicone and better still free of any volatile solvent.

The term “oil” means any non-aqueous medium which is liquid at room temperature (25° C.) and atmospheric pressure (760 mm Hg).

The term “non-volatile” medium means any medium capable of remaining on the skin or the lips for several hours. A non-volatile medium in particular has a non-zero vapour pressure, at room temperature and atmospheric pressure, of less than 0.02 mm Hg (2.66 Pa).

The term “volatile” means a medium which is capable of evaporating from the skin or the lips in less than one hour. A volatile medium is chosen in particular from media having a vapour pressure, at room temperature and atmospheric pressure, ranging from 0.02 mm to 300 mm Hg (2.66 Pa to 40 000 Pa) and better still ranging from 0.1 to 90 mm Hg (13 Pa to 12 000 Pa). In addition, the volatile silicones generally have a viscosity of less than 5 cSt at room temperature and atmospheric pressure.

The composition may contain one or more non-volatile hydrocarbon-based oils and one or more non-volatile silicone compounds.

The expression “inert particulate phase” means any filler which is solid at room temperature and atmospheric pressure, used alone or in combination, which does not react chemically with the other ingredients of the composition and which is insoluble in these ingredients, even when these ingredients are brought to a temperature above room temperature (for example the melting point of these ingredients).

This composition is, in particular, a cosmetic or dermatological composition. It thus contains ingredients that are compatible with keratin materials, i.e. the skin, the lips, keratin fibres and the nails. It can be in the form of an anhydrous gel, an oil-in-water or water-in-oil emulsion or dispersion, or alternatively in the form of a multiple emulsion. It can also be in a more or less fluid form, in the form of a paste, or in the form of a non-deformable or rigid solid, optionally cast as a stick or a dish. It is preferably in fluid or stick form, in particular anhydrous fluid or stick form. The term “fluid” means a composition which flows under its own weight, as opposed to a solid.

According to the invention, the non-volatile silicone compound and the non-volatile hydrocarbon-based oil are mutually compatible. The deposit obtained on the skin or the lips is homogeneous and soft. It leaves virtually no traces on a support which comes into contact with the deposit and does not migrate, especially in the wrinkles and fine lines around the lips.

Advantageously, the composition comprises an agent for dispersing solid particles which contains at least one non-volatile hydrocarbon-based compound which is compatible with the non-volatile hydrocarbon-based oil and incompatible with the non-volatile silicone compound, the dispersant having solubility parameters such that 16.40 (J/cm³)^{½}≦δ_{D}≦19.00 (J/cm³)^{½} and 2.00 (J/cm³)^{½}≦δₐ≦9.08 (J/cm³)^{½}. The non-volatile hydrocarbon-based compound is incompatible with the non-volatile silicone compound, that is to say that it is insoluble or indispersible in the non-volatile silicone compound.

The dispersant allows the dispersion of all the solid particles, at room temperature and atmospheric pressure, present in the composition, such as fillers, pigments and nacres.

The composition according to the present invention thus advantageously comprises one or more physiologically acceptable non-volatile hydrocarbon-based compounds, serving as dispersants for solid particles.

The composition advantageously contains at least one ingredient chosen from cosmetic and dermatological active agents and dyestuffs, and mixtures thereof. The incompatibility of the non-volatile silicone compound and of the non-volatile hydrocarbon-based dispersant makes it possible in particular to limit or prevent altogether the transfer of the composition and in particular the transfer of the active agents and/or dyestuffs. It is thus possible to keep these active agents and/or dyestuffs where they were deposited, while at the same time giving the composition wear-comfort properties due to the replacement, compared with the prior art, of the volatile solvents with non-volatile oils of low molecular mass. The non-volatile hydrocarbon-based oil of low molecular mass serves to compatibilize (dissolve or dispersed, and better still dissolve) the non-volatile silicone compound and the dispersant. In the absence of the non-volatile hydrocarbon-based oil of low molecular mass, the silicone compound and the dispersant form two phases that are immiscible, at elevated temperature and at room temperature.

In another embodiment, the present invention provides the use, in particular the cosmetic use, in a cosmetic composition or for the manufacture of a composition for topical application, of at least one non-volatile hydrocarbon-based oil with a molecular mass ranging from 230 to 420 g/Mol, of at least one non-volatile silicone compound which is compatible with the non-volatile hydrocarbon-based oil and of at least one inert particulate phase, for reducing or even preventing altogether the transfer of a film of composition deposited on the skin and/or the lips of a human being onto a support placed in contact with the film and/or for preserving the gloss of the film and/or for making this film comfortable to wear and/or for increasing the staying power of the film over time and/or for reducing the migration of the film.

In another embodiment, the present invention provides a cosmetic care process or make-up process for the lips, integuments or the skin, which consists in applying a cosmetic composition as defined above to the lips, integuments or the skin, respectively.

In another embodiment, the present invention provides a cosmetic process for limiting or even preventing altogether the transfer of a care or make-up composition for the skin or the lips onto a support other than the said skin and the said lips, and/or for limiting or even preventing altogether the migration of this composition, containing at least one ingredient chosen from dyestuffs and cosmetic and dermatological active agents and mixtures thereof, which consists in introducing into the said composition a combination of at least one non-volatile silicone compound, an inert particulate phase and at least one non-volatile hydrocarbon-based oil which is compatible with the non-volatile silicone compound, and optionally a non-volatile dispersant which is compatible (soluble or dispersible) with the non-volatile hydrocarbon-based oil and incompatible with the silicone compound, as defined above.

It has moreover been found that the compositions according to the present invention have particularly advantageous spreading and adhesion qualities on the skin and the lips, as well as a pleasant, creamy feel. The present compositions also have the advantage of being easy to remove, especially with a conventional make-up-removing milk. This is especially noteworthy since the compositions of the prior art with high “transfer-resistance” properties are very difficult to remove. In general, they are sold with a specific make-up-removing product, which places an additional constraint on the user.

The non-volatile silicone compounds of the invention must be soluble or dispersible in the non-volatile hydrocarbon-based oils and in particular in non-volatile esters with a molecular mass of from 230 to 420 g/Mol. They are preferably chosen from compounds that are liquid at room temperature and, even more preferably, they have a viscosity which is within the range from 5 to 1,000,000 cSt at 25° C., better still from 10 to 500,000 cSt and preferably from 10 to 5,000 cSt.

Examples of silicone compounds which may be mentioned are polydimethylsiloxanes, phenyltrimethicones, polyalkylmethylsiloxanes, silicone resins such as those disclosed in documents JP-A-62 61911, JP-A-61 65809 and EP-A-602 905 (which are incorporated herein by reference), and fluorosilicones, and mixtures thereof.

In particular, these silicone compounds are chosen from non-volatile polydimethylsiloxanes (PDMSs); polydimethylsiloxanes comprising alkyl, alkoxy or phenyl groups pendent or at the end of a silicone chain, these groups containing from 2 to 24 carbon atoms; phenyl trimethicones, phenyl dimethicones, phenyl trimethylsiloxydiphenylsiloxanes, diphenyl dimethicones, diphenyl methyldiphenyltrisiloxanes, 2-phenylethyl trimethylsiloxysilicates; fluorosilicones comprising a fluoro group pendent or at the end of a silicone chain and containing from 1 to 12 carbon atoms, all or some of the hydrogen atoms of which are substituted with fluorine atoms; silicone resins; silicone gums including dimethiconols; and mixtures thereof.

The content by mass of silicone compound in the final composition is suitably higher than 0.01% based on the total weight of the composition, for example, within the range from 0.5% to 90%, preferably from 5% to 60% and even more preferably from 10% to 50%, based on the total weight of the composition.

The non-volatile silicone compound is preferably present in the present compositions in a proportion equal to or greater than that of the non-volatile hydrocarbon-based compound, serving as dispersant, in other words the ratio R defined by:

$$R = \frac{\% by mass of non \text{-} volatile silicone compound}{\% by mass of non \text{-} volatile dispersant}$$

is preferably greater than or equal to 1.

The non-volatile hydrocarbon-based compounds of the invention, acting as dispersants for the solid particles, should also be compatible with the non-volatile hydrocarbon-based oils of low molecular mass, but, on the other hand, they should not be compatible with the silicone compounds described above.

These dispersants are fluid at room temperature and in particular liquid, and comprise in their chemical structure at least one nonionic polar group below:

—COOH;

monosubstituted or disubstituted —OH (primary or secondary);

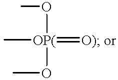

—NHR or —NR₁R₂, wherein R, R₁ and R₂ each independently represent a linear or branched C₁ to C₂₀ alkyl or alkoxy radical or R₁ and R₂ optionally form a ring, or

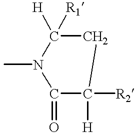

wherein R₁′ and R₂′ each independently may be equal to H or to a linear or branched C₁ to C₂₀ alkyl or alkoxy chain.

In particular, these dispersants may comprise one, two, three or more nonionic polar groups.

Preferably, these dispersants comprise at least one nonionic polar group below:

—COOH;

monosubstituted or disubstituted —OH (primary or secondary);

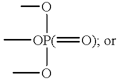

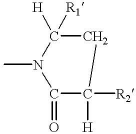

wherein R₁′ and R₂′ each independently may be equal to H or to a linear or branched C₁ to C₂₀ alkyl or alkoxy chain. These dispersants advantageously comprise at least one OH group.

The non-volatile hydrocarbon-based compounds serving as dispersants according to the invention are preferably such that their Hansen solubility parameters δ_{D}, δₚ, and δₕ are such that:

16.40 (J/cm³)^{½}≦δ_{D}≦19.00 (J/cm³)^{½} and preferably 16.70 (J/cm³)^{½}≦δ_{D}≦18.50 (3/cm³)^{½};

2.00 (J/cm³)^{½}≦δₐ≦9.08 (J/cm³)^{½} and preferably 4.00 (J/cm³)^{½}≦δₐ≦9.08 (J/cm³)^{½} and more preferably 5.00 (J/cm³)^{½}≦δₐ≦6.80 (J/cm³)^{½} given that δₐ=(δₚ²+δₕ²)^{½}

The definition of the Hansen solubility parameters is well known to those skilled in the art and is disclosed in particular in the article by C. M. Hansen: “The three dimensional solubility parameters”, J. Paint Technol., vol. 39, p. 105 (1967), which is incorporated herein by reference. These parameters are also described in document JP-A-08-109121 by Kao and the document by D. W. van Krevelen, Properties of Polymers, Elsevier, N.Y. (1990), p. 190, which are incorporated herein by reference.

According to this Hansen space:

δ_{D} characterizes the London dispersion forces arising from the formation of dipoles induced during molecular impacts;

δₚ characterizes the Debye forces of interaction between permanent dipoles; and

δₕ characterizes the specific forces of interaction (such as hydrogen bonding, acid/base, donor/acceptor, etc.).

The parameters δ_{D}, δₚ, and δₕ are generally expressed in (J/cm³)^{½}. They are determined at room temperature (25° C.) and in particular according to the calculation method indicated in JP-A-08-109121.

In the compositions according to the present invention, any non-volatile hydrocarbon-based fluid and in particular liquid dispersant or mixture of non-volatile hydrocarbon-based fluid dispersants which satisfies the above relationships can be used. In this case, the solubility parameters of the mixture are determined from those of the fluid dispersants taken separately, according to the following relationships:

$${δ}_{Dmel} = {\sum }_{i} \times i {δ}_{Di} ; {δ}_{pmel} = {\sum }_{i} \times i {δ}_{pi} and {δ}_{hmel} = {\sum }_{i} \times i {δ}_{hi}$$

where xi represents the volume fraction of the non-volatile hydrocarbon-based fluid dispersant (i) in the mixture, and “mel” means of the mixture.

It is within the capabilities of a person skilled in the art to determine the amounts of each non-volatile hydrocarbon-based fluid dispersant in order to obtain a mixture of non-volatile hydrocarbon-based fluid dispersants which satisfies the above relationships.

The molecular mass of the hydrocarbon-based fluid dispersants is greater than 600 g/Mol and preferably greater than 700 g/Mol.

As examples of non-volatile hydrocarbon-based dispersants which may be used in the invention, mention may be made of diisostearyl malate, certain monoesters or polyesters of polyols such as diglyceryl diisostearate or diglyceryl triisostearate or alternatively poly(12-hydroxystearic acid)s such as Solsperse 21 000 sold by the company Zeneca or Arlacel P 100 sold by the company Uniqema, and mixtures thereof. Diglyceryl triisostearate and poly(12-hydroxystearic acid)s are preferably used.

The content by mass of dispersant in the final composition is, for example, within the range from 2% to 40%, preferably from 2.5% to 20%, and better still from 3% to 10%, of the total weight of the composition.

As non-volatile hydrocarbon-based solvents or oils of low molecular mass which can be used in the invention, mention may be made of esters in the form of monoesters, diesters, and, in general, polyesters with a molecular mass ranging from 230 to 420 g/Mol.

The esters may be linear or branched, and saturated or unsaturated. Preferably, they are in branched and saturated form. These esters are preferably esters of C₂ to C₁₈ acid and in particular of C₂ to C₂₀ alcohol or of C₂ to C₈ polyol or mixtures thereof. As non-volatile solvents or oils which may be used in the invention, mention may be made of neopentanoic acid esters, for instance isodecyl neopentanoate (242.4), isotridecyl neopentanoate (270.44), isostearyl neopentanoate (354.62), and octyldodecyl neopentanoate (382.67); isononanoic acid esters, for instance isononyl isononanoate (284.48), octyl isononanoate (270.44), isodecyl isononanoate (298.51), isotridecyl isononanoate (340.59), and isostearyl isononanoate (410.73); and also isopropyl alcohol esters, such as isopropyl myristate (270.46), isopropyl palmitate (298.51), isopropyl stearate or isostearate; cetyl octanoate (368.64); tridecyl octanoate (326.55), PEG-4 diheptanoate (418.51) and 2-ethylhexyl palmitate (368.64); C₁₂-C₁₅ alkyl benzoate (309.04); neopentyl glycol diheptanoate (328.49); and propylene glycol diethyl 2-hexanoate. These esters are cited as their CTFA names (International Cosmetic Ingredient Dictionary, 5th edition et seq). Mention may also be made of alkanes, for instance isoeicosane (282.55). The figures in parentheses correspond to their molecular mass given in g/Mol.

Preferably, neopentanoic or isononanoic acid esters are used.

The non-volatile hydrocarbon-based solvent(s) of low molecular mass according to the present invention especially represent(s) a content by mass of from 5% to 99%, preferably from 10% to 60%, better still from 15% to 50%, and even better still from 15% to 30%, of the total mass of the composition.

The composition can also contain at least one additional fatty substance other than the non-volatile silicone compound, the non-volatile hydrocarbon-based solvent or oil and the non-volatile hydrocarbon-based dispersant compound, chosen from waxes, gums and fatty substances that are pasty at room temperature, oils and mixtures thereof, of mineral, animal, plant, or synthetic origin.

The additional oily fatty substances of the composition can be a cosmetically or dermatologically acceptable oil and in general a physiologically acceptable oil, chosen in particular from volatile and non-volatile oils of mineral, animal, plant, or synthetic origin.

As additional oils which can be used in the composition according to the invention, mention may be made in particular of:

hydrocarbon-based oils of animal origin, such as perhydrosqualene;

hydrocarbon-based plant oils such as liquid triglycerides of fatty acids of 4 to 24 carbon atoms, such as heptanoic or octanoic acid triglycerides or alternatively sunflower oil, corn oil, soybean oil, marrow oil, grapeseed oil, sesame or rape oil, hazelnut oil, apricot oil, macadamia oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter;

linear or branched hydrocarbons of mineral or synthetic origin, such as liquid paraffins and derivatives thereof, petroleum jelly, polydecenes, and hydrogenated polyisobutene, such as parleam;

synthetic esters and ethers in particular of fatty acids, such as the oils of formula R₃COOR₄ in which R₃ represents a higher fatty acid residue containing from 1 to 40 carbon atoms and R₄ represents a hydrocarbon-based chain containing from 1 to 40 carbon atoms with R₃ +R₄≧27, such as, for example, purcellin oil, 2-octyldodecyl stearate, 2-octyldodecyl erucate, or isostearyl isostearate; hydroxylated esters such as octyldodecyl hydroxystearate, triisocetyl citrate, and fatty alkyl heptanoates, octanoates and decanoates; polyol esters such as propylene glycol diisostearate; and pentaerythritol esters such as pentaerythrityl tetraisostearate;

fatty alcohols containing from 12 to 26 carbon atoms, such as octyldodecanol, 2-butyloctanol, 2-hexyldecanol, 2-undecylpentadecanol or oleyl alcohol;

partially hydrocarbon-based and/or silicone-based non-volatile fluoro oils such as methoxynonafluorobutane; and

mixtures thereof.

The additional non-volatile oil(s) of the composition can represent from 0.1 % to 90% by weight, preferably from 5% to 60% by weight, and better still from 10% to 50% by weight, of the total weight of the composition

Advantageously, the compositions of the present invention contain little or no volatile oil and, in particular, less than 5% by weight, better still less than 2% by weight, of the total weight of the composition, and in particular the present compositions contain no volatile oil.

The composition also contains an inert particulate phase which contains at least one absorbent or non-absorbent inert filler, that is to say one or more inert fillers which in particular absorb oils. Preferably, these fillers have an apparent diameter ranging from 0.01 to 150 μm. An apparent diameter corresponds to the diameter of the circle in which the elementary particle fits along its smallest dimension (thickness for leaflets).

The fillers may be mineral or organic, and lamellar, spherical, or oblong. Mention may be made of talc, mica, silica, kaolin, polyamide powders such as Nylon® powder (Orgasol® from Atochem), poly-β-alanine powder and polyethylene powder, polytetrafluoroethylene (Teflon®) powders, lauroyllysine, starch, boron nitride, hollow polymer microspheres such as those of polyvinylidene chloride/acrylonitrile, for instance Expancel® (Nobel Industrie), acrylic acid copolymers, (Polytrap® from Dow Corning) and silicone resin microbeads (Tospearl® from Toshiba, for example), precipitated calcium carbonate, magnesium carbonate and hydrocarbonate, hydroxyapatite, hollow silica microspheres (Silica Beads® from Maprecos) and glass or ceramic microcapsules, and mixtures thereof.

The inert particulate phase can represent from 0.1% to 30% by weight, better still from 2% to 25% by weight, and even better still from 10% to 20% by weight, of the total weight of the composition,

The respective amounts of non-volatile silicone compounds, of hydrocarbon-based oil of low molecular mass and of inert particulate phase are chosen in an amount which is sufficient to give the composition transfer-resistance, gloss and wear-comfort properties.

The composition of the invention can advantageously comprise one or more dyestuffs containing at least (one or more) pulverulent compounds and/or one or more liposoluble or water-soluble dyes, for example in a proportion of from 0% to 70% by weight, and in particular from 0.01% to 70% by weight, relative to the total weight of the composition. The pulverulent compound(s) may be chosen from the pigments and nacres usually used in cosmetic or dermatological compositions, and mixtures thereof. The pulverulent dye compounds advantageously represent up to 50% by weight, for example from 0.01% to 50% by weight, and better still from 1% to 40% by weight, of the weight of the composition.

The pigments may be white or coloured, mineral and/or organic, interferential or non-interferential, insoluble in the liquid fatty phase, and intended to colour and/or opacify the composition. Among the mineral pigments which may be mentioned are titanium dioxide, optionally surface-treated, zirconium oxide, zinc oxide or cerium oxide, as well as zinc oxide, iron oxide or chromium oxide, manganese violet, ultramarine blue, chromium hydrate and ferric blue. Among the organic pigments which may be mentioned are carbon black, pigments of D & C type and lakes based on cochineal carmine or based on barium, strontium, calcium or aluminium.

The nacreous pigments may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the abovementioned type, as well as nacreous pigments based on bismuth oxychloride.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow. They can represent from 0% to 20% by weight, and in particular 0.01% to 20% by weight, and better still from 0.1% to 6% by weight, of the total weight of the compositions. The water-soluble dyes are, for example, beetroot juice or methylene blue and can represent up to 6% by weight of the total weight of the composition.

The composition of the invention can also contain one or more cosmetic or dermatological active agents such as those conventionally used.

As cosmetic or dermatological active agents which can be used in the composition of the invention, mention may be made of moisturizers, vitamins, essential fatty acids, sphingolipids, sunscreens, and calmants (for example bisabolol). These active agents are used in an amount which is usual for those skilled in the art and in particular at concentrations of from 0% to 20% by weight, in particular from 0.001% to 20% by weight, and better still from 0.1% to 5% by weight, relative to the total weight of the composition. Depending on the type of application envisaged, the composition according to the invention can furthermore comprise the constituents conventionally used in the fields under consideration, which are present in an amount which is suitable for the desired presentation form.

Among the waxes that are solid at room temperature, which may be present in the composition according to the invention, mention may be made of hydrocarbon-based waxes such as optionally modified beeswax, carnauba wax, candelilla wax, ouricury wax, Japan wax, cork fibre wax, sugar cane wax, paraffin wax, lignite wax, microcrystalline waxes, lanolin wax, montan wax, ozokerites, polyethylene waxes, the waxes obtained by Fischer-Tropsch synthesis, and C₂₀-C₆₀ fatty alcohols. It is also possible to use silicone waxes, among which mention may be made of alkyl- and alkoxypolymethylsiloxanes and/or polymethylsiloxane esters, and mixtures thereof.

The waxes may be present in a proportion of 0-50% by weight (for example from 0.01% to 50% by weight) in the composition and better still from 5 % to 20% by weight, so as not to excessively reduce the gloss of the composition and of the film deposited on the lips and/or the skin.

Pasty fatty substances which may be mentioned are fatty substances with a melting point ranging from 25° C. to 45° C. and/or a viscosity at 40° C. ranging from 0.1 Pa.s to 40 Pa.s, measured using a Contraves TV viscometer equipped with an MS-r3 or MS-r4 spindle spinning at 60 Hz. Examples of pasty fatty substances which may be mentioned are PDMSs with pendent chains of the alkyl or alkoxy type containing from 8 to 24 carbon atoms, for instance stearyldimethicone; esters of fatty alcohol or of fatty acid, for instance cholesterol esters, polyvinyl laurate, arachidyl propionate; PVP/eicosene copolymer; lanolins and derivatives thereof such as acetylated lanolins or oxypropylenated lanolins, and mixtures thereof.

The nature and amount of the waxes, pasty fatty substances and gums depend on the desired mechanical properties and textures.

The composition can also comprise any additive usually used in such compositions, such as thickeners (hectorite modified with distearyldimethylammonium chloride, for example, which is known under the name Bentone®), antioxidants, fragrances, preserving agents, surfactants, liposoluble polymers, for instance polyalkylenes, in particular polybutene, polyacrylates and silicone polymers that are compatible with the fatty phase, as well as polyvinylpyrrolidone derivatives. Needless to say, a person skilled in the art will take care to select this or these optional additional compound(s), and/or the amount thereof, such that the advantageous properties of the composition according to the invention are not, or are not substantially, adversely affected by the envisaged addition.

The compositions according to the present invention can be prepared in the usual manner by a person skilled in the art. They can be in the form of a cast product and, for example, in the form of a stick or tube, or in the form of a dish which can be used by direct contact or with a sponge or alternatively in a boiling pan. In particular, they find an application as cast foundations, cast blushers or eyeshadows, lipsticks, care bases or care balms for the lips and concealer products. They can also be in the form of a soft paste or alternatively a gel or a more or less fluid cream. In this case, they can constitute foundations or lipsticks, lip glosses, suncare products or skin-colouring products.

The compositions of the invention are advantageously anhydrous and can contain less than 5% by weight of water relative to the total weight of the composition. In this case, they can be in particular in the form of an oily gel, an oily liquid, a paste or a stick or alternatively in the form of a vesicular dispersion containing ionic and/or nonionic lipids. They can also be in the form of a simple or multiple emulsion containing an oily or aqueous continuous phase, or an oily dispersion in an aqueous phase by means of vesicles containing ionic and/or nonionic lipids. These presentation forms are prepared according to the usual methods of the fields under consideration.

These compositions for topical application can in particular constitute a cosmetic or dermatological protective, treatment or care composition for the face, for the neck, for the hands or for the body (for example a care cream, antisun oil or body gel), a make-up composition (for example a make-up gel, cream or stick) or an artificial tanning composition or skin-protecting composition.

As noted above, the present invention also provides novel methods for caring for and/or making up the skin, the lips, and/or the nails by applying an effective amount of a composition according to the present invention to the skin, lips, and/or nails. Of course the amount of the composition applied and the schedule of applying the composition will depend on the exact effect desired to be achieved. Thus, when using the present compositions for making up the skin, the lips, and/or the nails, the composition will be applied in an amount sufficient to achieve the desired level of make up and will be applied as desired by the user. On the other hand, when using the present composition to care for the skin, the lips, and/or the nails, the compositions are suitably applied to the skin, the lips, and/or the nails in an amount of 0.1 to 20 mg/cm², preferably 0.3 to 10 mg/cm², more preferably 1 to 5 mg/cm² (these numbers being approximate). The amount is generally 1 or 2 mg/cm². These quantities correspond to make-up products as well as sun-care products. The exact quantity will depend on the desired result on the skin, lips of ends of the body (e.g., hair, nails, eyelashes, etc.). For example, with a shiny product such as a lip product, more product will be typically applied to obtain the desired shiny affect. On the other hand, with a non-transferred product, less product will be typically applied to obtain less transfer. In the examples described below, the quantity applied to the lips is about 2 mg/cm².

The composition may be applied to the skin, the lips, and/or the nails in a regime which includes application of the composition weekly, every other day, daily, or twice daily. The application of the composition to the skin, the lips, and/or the nails may be continued until the desired degree of improvement is achieved or continued indefinitely for preventative purposes.

Other features of the present invention will be come apparent in the course of the following description of exemplary embodiments which are given for illustration of the invention and are not intended to be limiting thereof.

### EXAMPLES

In the following examples, all percentages are percentages by weight based on the total weight of the composition. The names of certain ingredients are given as the CTFA name.

### Example 1

### Lipstick In Stick Form

| | |
|---|---|
| Phase A | |
| Poly(12-hydroxystearic acid) sold under the | 3.14% |
| reference Solsperse 21 000 by the company Zeneca | |
| Bis diglyceryl poly(2-acyl adipate) | 3.13% |
| Lanolin | 2.09% |
| Arachidyl propionate | 6.46% |
| Octyldodecyl neopentanoate | 7.84% |
| Antioxidant | 0.06% |
| Phase B | |
| Isononyl isononanoate | 20.68% |
| Bentone 38V sold and/or manufactured by the | 1.32% |
| company SASI | |
| Phase C | |
| Ozokerite | 1.8% |
| Polyethylene wax (Mₙ* = 500) | 6.0% |
| Polyethylene wax (Mₙ* = 400) | 3.0% |
| Phase D | |
| Titanium dioxide | 2.79% |
| DC Red No. 7 | 0.06% |
| Black iron oxide | 0.34% |
| Red iron oxide | 0.84% |
| Lauroyllysine | 5.00% |
| Kaolin | 10.43% |
| Phase E | |
| Phenyltrimethicone (1000 cSt) | 15.60% |
| Phenyltrimethicone (20 cSt) | 7.62% |
| Phase F | |
| Bisabolol | 0.40% |
| Phase G | |
| Nacres | 1.40% |
| | 100% |
| | by mass |

*Mₙmeans the number-average molecular mass.

Procedure

Phase A is prepared by successively weighing out the constituents and mixing them together with stirring at 80 to 90° C. Phase B is prepared by dispersing the Bentone in the hydrocarbon-based oil. Phases A & B are then mixed together at 80 to 90° C.

The particulate phase D is ground into a fraction of phases A+B using a three-roll mill.

The waxy phase C is added and the mixture is heated at 100° C. until the waxes have completely dissolved. The silicones of phase E are next added, followed by phase F and finally the nacres of phase G, at 100° C.

The final mixture obtained can then be cast at 100° C. in moulds to obtain sticks.

This lipstick in stick form gives a satin to gloss effect, is comfortable to wear over time, non-greasy and non-sticky, and has good transfer-resistance properties.

| | No.2 | No.3 | No.4 | No.5 | No.6 |
|---|---|---|---|---|---|
| | (invention) | (comparative) | (comparative) | (comparative) | (comparative) |
| Phase A | | | | | |
| - Poly(12-hydroxystearic acid) | 3.00% | 3.00% | 3.00% | — | — |
| - Castor oil | — | — | — | — | 3.00% |
| - Bis diglyceryl poly(2-acyl adipate) | 3.00% | 3.00% | 3.00% | 3.00% | 3.00% |
| - Lanolin | 2.00% | 2.00% | 2.00% | 2.00% | 2.00% |
| - Arachidyl propionate | 6.18% | 6.18% | 6.18% | 6.18% | 6.18% |
| - Octyldodecyl neopentanoate | 7.50% | — | 7.50% | 7.50% | 7.50% |
| - Hydrogenated polyisobutene(Mₙ = 450.89) sold | — | 7.50% | — | 3.00% | — |
| under the reference Parleam by the | | | | | |
| company Nippon Oil Fats | | | | | |
| - Antioxidant | 0.06% | 0.06% | 0.06% | 0.06% | 0.06% |
| Phase B | | | | | |
| - Isononyl isononanoate | 20.68% | — | 20.68% | 20.68% | 20.68% |
| - Hydrogenated polyisobutene(Mₙ = 450.89) sold | — | 20.68% | — | — | — |
| under the reference Parleam by Nippon Oil Fats | | | | | |
| - Bentone 38V | 1.32% | 1.32% | 1.32% | 1.32% | 1.32% |
| Phase C | | | | | |
| - Ozokerite | 2.00% | 2.00% | 2.00% | 2.00% | 2.00% |
| - Polyethylene wax (Mₙ = 500) | 6.67% | 6.67% | 6.67% | 6.67% | 6.67% |
| - Polyethylene wax (Mₙ = 400) | 3.33% | 3.33% | 3.33% | 3.33% | 3.33% |
| Phase D | | | | | |
| - Titanium dioxide | 0.33% | 0.33% | 0.33% | 0.33% | 0.33% |
| - DC Red 27 Al Lake | 0.43% | 0.43% | 0.43% | 0.43% | 0.43% |
| - DC Red 7 | 0.57% | 0.57% | 0.57% | 0.57% | 0.57% |
| - FD and C Yellow 6 Al Lake | 0.17% | 0.17% | 0.17% | 0.17% | 0.17% |
| - Lauroyllysine | 5.00% | 5.00% | 5.00% | 5.00% | 5.00% |
| - Kaolin | 13.00% | 13.00% | 13.00% | 13.00% | 13.00% |
| Phase E | | | | | |
| - Phenyltrimethicone (1000 cSt) | 15.60% | 15.60% | — | 15.60% | 15.60% |
| - Polybutene sold under the reference Dynapak H | — | — | 14.63% | — | — |
| 100 by the company Pakhoed Products | | | | | |
| - Phenyltrimethicone (20 cSt) | 7.62% | 7.62% | — | 7.62% | 7.62% |
| - Hydrogenated polyisobutene(Mₙ =450.89) sold | — | — | 8.59% | — | — |
| under the reference Parleam by Nippon Oil Fats | | | | | |
| Phase F | 0.40% | 0.40% | 0.40% | 0.40% | 0.40% |
| - Bisabolol | | | | | |
| Phase G | 1.14% | 1.14% | 1.14% | 1.14% | 1.14% |
| - Nacres | | | | | |

100.00% by mass

Procedure

Examples 2 to 6 were performed according to the same procedure as that described for Example 1.

The sensory properties of Comparative Examples 3, 4, 5 and 6 were compared with those of Example 2 according to the present invention.

### Comparison

### Example 2 and Example 3

The esters of low molecular mass, namely isononyl isononanoate and octyldodecyl neopentanoate of Example 2, were replaced in Example 3 with hydrogenated polyisobutene whose molecular mass is 450.89 g/Mol.

The transfer-resistance properties of the composition of Example 2 were judged to be superior to those of the composition of Example 3. They were evaluated 15 minutes after applying lipstick, by kissing a sheet of paper (kiss test).

The esters of low molecular masses are thus favourable to transfer resistance. The lipstick of Example 2 was judged to give a satin to gloss effect and to be comfortable.

### Comparison

### Example 2 and Example 4

The silicones, namely the phenyltrimethicones (20 and 1000 cSt) of Example 2, were replaced with polybutene and hydrogenated polyisobutene in Example 4. The proportions of these alkanes were chosen so as to obtain a viscosity close to that of the silicone mixture.

The transfer-resistance properties of the composition of Example 2 were judged to be superior to those of the composition of Example 4. On the other hand, this latter composition gave a film which was too oily.

The non-volatile silicones are thus favourable to transfer resistance, without making the lips greasy.

### Comparison

### Example 2 and Example 5

The dispersant (Solsperse 21 000) of Example 2 was replaced in Example 5 with hydrogenated polyisobutene whose molecular mass (450.89 g/Mol) is greater than that of the hydrocarbon-based oils of low molecular mass of the invention, and the Hansen solubility parameters 8D and ⁶a are, respectively, 15.48 and 0 (J/cm³)^{½}.

The composition of Example 5 gives a film on the lips which is less glossy than that of the composition of Example 2 for a similar transfer.

The dispersant of the invention is thus favourable to the gloss of the film for comparable transfer-resistance properties.

### Comparison

### Example 2 and Example 6

The dispersant Solsperse 21 000 of Example 2 was replaced in Example 6 with castor oil, whose solubility parameter δₐ is 9.09 (J/cm³)^{½}.

Example 6 gives a film on the lips which is less glossy than that of the composition of Example 2 for a similar transfer.

The dispersant of the invention is thus favourable to the gloss of the film for comparable transfer-resistance properties.

Examples 2 and 6 clearly show that the combination: non-volatile hydrocarbon-based oil/non-volatile silicone compound/fillers optionally containing an agent for dispersing solid particles, gives the composition transfer-resistance properties that are better than those of the prior art, without harming the gloss properties, and at the same time being comfortable to wear.

### Example 7

### Lipstick In Stick Form (comparative example)

| | |
|---|---|
| Phase A | |
| Poly(12-hydroxystearic acid) | 3.00% |
| Bis diglyceryl poly(2-acyl adipate) | 3.00% |
| Lanolin | 2.00% |
| Arachidyl propionate | 6.18% |
| Antioxidant | 0.06% |
| Phase B | |
| Ozokerite | 2.00% |
| Polyethylene wax (Mₙ = 500) | 6.67% |
| Polyethylene wax (Mₙ = 400) | 3.33% |
| Phase C | |
| Titanium dioxide | 0.33% |
| DC Red 27 Al Lake | 0.43% |
| DC Red 7 | 0.57% |
| FD and C Yellow 6 Al Lake | 0.17% |
| Lauroyllysine | 5.00% |
| Bentone 38V | 1.32% |
| Phase D | |
| Kaolin | 13.00% |
| Phase E | |
| Phenyltrimethicone (1000 cSt) | 15.60% |
| Phenyltrimethicone (20 cSt) | 7.62% |
| Phase F | |
| Bisabolol | 0.40% |
| Phase G | |
| Nacres | 1.14% |
| Phase H | |
| Cyclotetradimethylsiloxane (D4) | 28.18% |
| | 100.00% |
| | by mass |

Procedure

Phase A is prepared by successively weighing out the constituents and mixing them together with stirring at 80 to 90° C.

The coloured particulate phase C is ground into phase A using a three-roll mill.

Phase E is prepared by successively weighing out the constituents and mixing them together with stirring.

The inert particulate phase D is ground into phase E using a three-roll mill.

The mixtures A+C and D+E are then combined and the waxy phase B is added. The mixture is heated at 100° C. until the waxes have completely dissolved.

Phase F is then added, followed by phase G and finally phase H, this last phase being introduced at 90° C.

The final mixture is then cast in suitable moulds in order to obtain sticks.

### Comparison

### Example 2 and Example 7

The esters of low molecular mass, namely isononyl isononanoate and octyldodecyl neopentanoate of Example 2, were replaced in Example 7 with a volatile silicone (D4).

The composition of Example 7 was judged to be very rich when applied and gave a film which was very matt and adhered strongly to the lips, unlike the composition of Example 2. The hydrocarbon-based oils of low molecular mass are thus favourable to the gloss and wear comfort.

| | |
|---|---|
| Phase A | |
| Diglyceryl triisostearate, sold under the reference | 3.00% |
| Salacos 43 by the company Nisshin Oil Mills | |
| Bis diglyceryl poly(2-acyl adipate) | 3.00% |
| Lanolin | 2.00% |
| Arachidyl propionate | 6.18% |
| Octyldodecyl neopentanoate | 7.50% |
| Antioxidant | 0.06% |
| Phase B | |
| Isononyl isononanoate | 20.68% |
| Bentone 38V | 1.32% |
| Phase C | |
| Ozokerite | 2.00% |
| Polyethylene wax (Mₙ = 500) | 6.67% |
| Polyethylene wax (Mₙ = 400) | 3.33% |
| Phase D | |
| Titanium dioxide | 0.33% |
| DC Red 27 Al Lake | 0.43% |
| DC Red 7 | 0.57% |
| FD and C Yellow 6 Al Lake | 0.17% |
| Lauroyllysine | 5.00% |
| Kaolin | 13.00% |
| Phase E | |
| Phenyltrimethicone (1000 cSt) | 15.60% |
| Phenyltrimethicone (20 cSt) | 7.62% |
| Phase F | 0.40% |
| Bisabolol | |
| Phase G | |
| Nacres | 1.14% |
| | 100.00% by mass |

Procedure

The composition of Example 8 is prepared according to the same procedure as that of Example 1. The gloss, wear-comfort and transfer-resistance properties are comparable to those of the composition of Example 2.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A care or make-up composition for keratin materials, comprising:
(a) at least one non-volatile hydrocarbon-based oil with a molecular mass ranging from 230 to 420 g/Mol;
(b) at least one non-volatile silicone compound which is compatible with the non-volatile hydrocarbon-based oil; and
(c) an inert particulate phase.

2. The composition according to claim 1, wherein said non-volatile hydrocarbon-based oil has a molecular mass ranging from 240 to 350 g/Mol.

3. The composition according to claim 1, wherein said non-volatile hydrocarbon-based oil has a molecular mass ranging from 240 to 300 g/Mol.

4. The composition according to claim 1, wherein said non-volatile hydrocarbon-based oil has a molecular mass ranging from 240 to 280 g/Mol.

5. The composition according to claim 1, wherein said non-volatile hydrocarbon-based oil is an ester.

6. The composition according to claim 1, wherein said non-volatile hydrocarbon-based oil is an ester of a C₂ to C₁₈ acid.

7. The composition according to claim 1, wherein said non-volatile hydrocarbon-based oil is selected from the group consisting of esters of C₂ to C₂₀ alcohols and esters of C₂ to C₈ polyols, and mixtures thereof.

8. The composition according to claim 1, wherein said non-volatile hydrocarbon-based oil is a branched acid ester.

9. The composition according to claim 1, wherein said non-volatile hydrocarbon-based oil is selected from the group consisting of neopentanoic acid esters, isononanoic acid esters, and mixtures thereof.

10. The composition according to claim 1, wherein said non-volatile hydrocarbon-based oil is selected from the group consisting of isodecyl neopentanoate, isotridecyl neopentanoate, isostearyl neopentanoate, octyldodecyl neopentanoate, isononyl isononanoate, octyl isononanoate, isodecyl isononanoate, isotridecyl isononanoate, isostearyl isononanoate, and mixtures thereof.

11. The composition according to claim 1, further comprising a dispersant, wherein said dispersant comprises at least one non-volatile hydrocarbon-based compound which is compatible with said non-volatile hydrocarbon-based oil and is incompatible with said non-volatile silicone compound.

12. The composition according to claim 11, wherein said dispersant has solubility parameters such that 16.40 (J/cm³)^{½}≦δ_{D}≦19.00 (J/cm³)^{½} and 2.00 (J/cm³)^{½}≦δₐ≦9.08 (J/cm³)^{½}.

13. The composition according to claim 11, wherein said dispersant has solubility parameters such that 16.70 (J/cm³)^{½}≦δ_{D}≦18.50 (J/cm³)^{½}.

14. The composition according to claim 11, wherein said dispersant has solubility parameters such that 4.00 (J/cm³)^{½}≦δₐ≦9.08 (J/cm³)^{½}.

15. The composition according to claim 11, wherein said dispersant has solubility parameters such that 5.00 (J/cm³)^{½}≦δₐ≦6.80 (J/cm³)^{½}.

16. The composition according to claim 11, wherein said dispersant has a molar mass greater than 600 g/Mol.

17. The composition according to claim 11, wherein said dispersant has a molar mass greater than 700 g/Mol.

18. The composition according to claim 11, wherein said dispersant has a chemical structure comprising at least one nonionic polar group selected from the group consisting of —COOH; —OH; 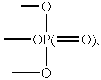
—NHR, wherein R represents a linear or branched C₁ to C₂₀ alkyl or alkoxy radical; —NR₁R₂ wherein R₁ and R₂ each independently represents a linear or branched C₁ to C₂₀ alkyl or alkoxy radical or R₁ and R₂ together can form a ring; and 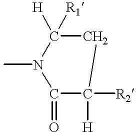
wherein R₁′ and R₂′ each independently may be equal to H or to a linear or branched C₁ to C₂₀ alkyl or alkoxy chain.

19. The composition according to claim 11, wherein said non-volatile hydrocarbon-based compound has a chemical structure comprising at least one nonionic polar group selected from the group consisting of —COOH; —OH; 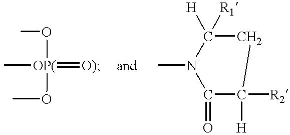
wherein R₁′ and R₂′ each independently may be equal to H or to a linear or branched C₁ to C₂₀ alkyl or alkoxy chain.

20. The composition according to claim 11, wherein said non-volatile hydrocarbon-based compound is selected from the group consisting of diisostearyl malate, polyol monoesters and polyesters and poly(12-hydroxystearic acids), and mixtures thereof.

21. The composition according to claim 11, wherein said dispersant is present in an amount by mass ranging from 2% to 40%, based on the total weight of said composition.

22. The composition according to claim 11, wherein said dispersant is present in an amount by mass ranging from 2.5% to 20%, based on the total weight of said composition.

23. The composition according to claim 11, wherein said dispersant is present in an amount by mass ranging from 3% to 10%, based on the total weight of said composition.

24. The composition according to claim 1, wherein said non-volatile silicone compound is a compound which is liquid at room temperature.

25. The composition according to claim 1, wherein said non-volatile silicone compound has a viscosity within the range from 5 to 1,000,000 cSt at 25° C.

26. The composition according to claim 1, wherein said non-volatile silicone compound has a viscosity within the range from 10 to 500,000 cSt.

27. The composition according to claim 1, wherein said non-volatile silicone compound has a viscosity within the range from from to 5,000 cSt.

28. The composition according to claim 1, wherein said non-volatile silicone compound is selected from the group consisting of non-volatile polydimethylsiloxanes (PDMSs); polydimethylsiloxanes comprising alkyl, alkoxy or phenyl groups that are pendent or at the end of a silicone chain, these groups containing from 2 to 24 carbon atoms; phenyl trimethicones, phenyl dimethicones, phenyl trimethylsiloxydiphenylsiloxanes, diphenyl dimethicones, diphenyl methyldiphenyltrisiloxanes and 2-phenylethyl trimethylsiloxysilicates;
fluorosilicones comprising a fluoro group which is pendent or at the end of a silicone chain and containing from 1 to 12 carbon atoms, all or some of the hydrogen atoms of which are replaced with fluorine atoms; silicone resins; silicone gums; dimethiconols; and mixtures thereof.

29. The composition according to claim 1, wherein said non-volatile silicone compound is present in an amount by mass of from 0.5% to 90%, based on the total mass said composition.

30. The composition according to claim 1, wherein said non-volatile silicone compound is present in an amount by mass of from 5% to 60%, based on the total mass of said composition.

31. The composition according to claim 1, wherein said non-volatile silicone compound is present in an amount by mass of from 10% to 50%, based on the total mass of said composition.

32. The composition according to claim 1, wherein said non-volatile hydrocarbon-based oil is present in an amount by mass of from 5% to 99%, based on the total mass of said composition.

33. The composition according to claim 1, wherein said non-volatile hydrocarbon-based oil is present in an amount by mass of from 10% to 60%, based on the total mass of said composition.

34. The composition according to claim 1, wherein said non-volatile hydrocarbon-based oil is present in an amount by mass of from 15% to 50%, based on the total mass of said composition.

35. The composition according to claim 1, further comprising at least one ingredient selected from the group consisting of cosmetic active agents, dermatological active agents and dyestuffs, and mixtures thereof.

36. The composition according to claim 11, wherein the ratio by mass of said non-volatile silicone compound relative to said dispersant is greater than or equal to 1.

37. The composition according to claim 1, further comprising at least one fatty substance other than the non-volatile silicone compound, the non-volatile hydrocarbon-based oil and the dispersant, which is selected from the group consisting of waxes, gums, fatty substances that are pasty at room temperature, and oils, and mixtures thereof.

38. The composition according to claim 35, wherein said dyestuffs comprise at least one pulverulent dye compound chosen from pigments and nacres, and mixtures thereof.

39. The composition according to claim 34, wherein said pulverulent dye compound is present in an amount up to 50% by weight of the total weight of said composition.

40. The composition according to claim 1, wherein said particulate phase contains at least one absorbent or non-absorbent inert filler.

41. The composition according to claim 40, wherein said inert filler is selected from the group consisting of spherical fillers, lamrellar fillers, oblong fillers, and mixtures thereof.

42. The composition according to claim 40, wherein said inert filler is selected from the group consisting of talc, mica, silica, kaolin , polyamide powders, poly-β-alanine powder, polyethylene powder, polytetrafluoroethylene powders, lauroyllysine, starch, boron nitride, hollow polymer micro spheres, acrylic acid copolymers, silicone resin microbeads, precipitated calcium carbonate, magnesium carbonate, magnesium hydrocarbonate, hydroxyapatite, hollow silica micro spheres, glass microcapsules, ceramic microcapsules, and mixtures thereof.

43. The composition according to claim 40, wherein said inert particulate phase is present in an amount of from 0.1% to 30% by weight of the total weight of said composition.

44. The composition according to claim 40, wherein said inert particulate phase is present in an amount of from 2% to 25%, by weight of the total weight of said composition.

45. The composition according to claim 40, wherein said inert particulate phase is present in an amount of from 10% to 20%, by weight of the total weight of said composition.

46. The composition according to claim 1, which is free of a volatile silicone.

47. The composition according to claim 1, which is free of a volatile solvent.

48. The composition according to claim 1, which is in the form of a stick or tube, in the form of a soft paste, in the form of a dish, an oily gel, an oily liquid, a vesicular dispersion containing ionic and/or nonionic lipids, or a water-in-oil or oil-in-water emulsion.

49. The composition according to claim 1, which is in anhydrous form.

50. The composition according to claim 1, which is a make-up composition.

51. The composition according to claim 1, which is in the form of a foundation, a blusher, an eyeshadow, a lipstick, a care base or care balm for the lips, a concealer product, an eyeliner or a mascara.

52. A lipstick, comprising:
(a) at least one non-volatile hydrocarbon-based oil with a molar mass ranging from 230 to 420 g/Mol;
(b) at least one non-volatile silicone compound which is soluble or dispersible in the non-volatile hydrocarbon-based oil,;and
(c) an inert particulate phase.

53. A method for caring for or making-up the lips or the skin, said method comprising applying to the lips or the skin a cosmetic composition, said cosmetic composition comprising:
(a) at least one non-volatile hydrocarbon-based oil with a molar mass ranging from 230 to 420 g/Mol;
(b) at least one non-volatile silicone compound which is soluble or dispersible in the non-volatile hydrocarbon-based oil,;and
(c) an inert particulate phase.

54. The method according to claim 53, wherein said composition further comprises a dispersant, said deispersant comprising at least one non-volatile hydrocarbon-based compound with solubility parameters such that 16.40 (J/cm³)^{½}≦δ_{D}≦19.00 (J/cm³)^{½} and 2.00 (J/cm³)^{½}≦δₐ≦9.08 (J/cm³)^{½}.

55. A method for reducing or even preventing altogether the transfer of a film of composition deposited on the skin and/or the lips of a human being to a support placed in contact with the film and/or for preserving its gloss and/or for making this film comfortable to wear and/or for increasing the staying power of the film over time and/or for reducing its migration, said method comprising applying to the lips or the skin a cosmetic composition to form said film, said cosmetic composition comprising:
(a) at least one non-volatile hydrocarbon-based oil with a molar mass ranging from 230 to 420 g/Mol;
(b) at least one non-volatile silicone compound which is soluble or dispersible in the non-volatile hydrocarbon-based oil,;and
(c) an inert particulate phase.

56. The method according to claim 55, wherein said composition is free of a volatile solvent.

57. The method according to claim 55, characterized in that the composition also contains a dispersant comprising at least one non-volatile hydrocarbon-based compound with solubility parameters such that 16.40 (J/cm³)^{½}≦δ_{D}≦19.00 (J/cm³)^{½} and 2.00 (J/cm³)^{½}≦δₐ≦9.08 (J/cm³)^{½}.
